(19) United States
(12) Patent Application Publication
(10) Pub. No.: US 2014/0206579 A1
(43) Pub. Date: Jul. 24, 2014

(54) DNA LIBRARIES ENCODING FRAMEWORKS WITH SYNTHETIC CDR REGIONS

(71) Applicant: Syndecion, LLC, Highland, MD (US)

(72) Inventors: Alan D. KING, Highland, MD (US); Stephen B. DEITZ, Catonsville, MD (US)

(73) Assignee: Syndecion, LLC, Highland, MD (US)

(21) Appl. No.: 13/971,184

(22) Filed: Aug. 20, 2013

Related U.S. Application Data
(60) Provisional application No. 61/742,882, filed on Aug. 21, 2012.
(60) Provisional application No. 61/742,883, filed on Aug. 21, 2012.

(51) Int. Cl. C07K 16/18

(52) U.S. Cl. CPC C07K 16/18
USPC 506/17; 506/26; 536/23.53

(57) ABSTRACT

A synthetic DNA library or a member of a synthetic DNA library of antibodies or fragments of antibody molecules having heavy chain(s) and lacking light chain(s) is described wherein the CDR within variable domains are synthesized using randomly assembled trinucleotide phosphoramidites (trimer phosphoramidites) to eliminate unwanted cysteine amino acids and/or stop codons.

### CROSS-REFERENCES TO RELATED APPLICATIONS

This application claims the priority benefit under 35 U.S.C. §119(e) of U.S. Provisional Application Ser. No. 61/742,882, entitled “DNA LIBRARIES ENCODING FRAMEWORKS WITH SYNTHETIC CDR REGIONS” filed Aug. 21, 2012, and U.S. Provisional Application Ser. No. 61/742,883, entitled “COMPLEX OF NON-COVALENTLY BOUND PROTEIN WITH ENCODING NUCLEIC ACIDS AND USES THEREOF” filed Aug. 21, 2012, both of which are incorporated herein by reference in their entirety.

### BACKGROUND OF THE INVENTION

1. Field of the Invention

The present invention is related to the field of biotechnology. More specifically, the invention is directed to single domain antibody development, synthesis, and methods of use.

2. Description of the Background

Antibody fragments comprised of the variable region of single domain heavy chain antibodies are called VHH fragments or VHH antibodies. Two methods of creating antigen specific VHH antibodies have been used. One is to vaccinate camelids and clone the VHH antibodies from camelid B cells (Muyldermans and Lauwereys 1999). Another is to clone VHH antibodies from B cells from naïve (unvaccinated) camelids and to improve specificity and affinity by random mutations and gene shuffling (van der Linden et al. 2000). _None of these methods used completely synthetic variable region sequences.

U.S. Pat. No. 5,759,808 describes single domain antibodies with two heavy chains each containing a single domain. It also describes obtaining the antibody genes from B cells of camelids. U.S. Pat. No. 5,800,988, U.S. Pat. No. 5,840,526, U.S. Pat. No. 6,005,079, U.S. Pat. No. 6,015,695, U.S. Pat. No. 6,765,087, U.S. Pat. No. 7,722,871, and U.S. Pat. No. 7,786,047, similarly require two heavy chains. This series of patents describe the use of VHH genes obtained from B cells of vaccinated camelids. They do not mention VHH antibodies with fully synthetic CDR regions or VHH antibodies with only one heavy chain.

U.S. Pat. No. 6,399,763 describes a VHH expression library wherein natural sequences are enhanced by introducing mutations or by shuffling of variable (CDR) regions. The source of antibody sequences is from unvaccinated camelids. U.S. Pat. No. 7,196,187 similarly claims synthetic or semisynthetic VHH antibodies derived from a naïve (unvaccinated) library. Introduction of mutations and gene shuffling are the techniques disclosed. Both of these patents require starting from natural sequences. They do not disclose VHH antibodies with fully synthetic CDR regions.

### SUMMARY OF THE INVENTION

One embodiment of this invention provides a method for development and synthesis of single domain antibodies using completely synthetic DNA sequences in the variable (CDR) regions of the single domain antibodies. Previous methods of obtaining libraries of single domain antibodies required starting with antibody sequence libraries obtained from B cells of live animals. The embodiment of this invention eliminates that need by using completely synthetic sequences.

For the synthetic sequences, it is inefficient to simply use random nucleotides in the synthesis of variable (CDR) regions because the percent of stop codons in the sequence would be statistically high enough to make the yield of full protein impractically low. This invention provides a method for eliminating unwanted stop codons in the random variable (CDR) region sequences by synthesizing variable regions using trinucleotide phosphoramidites, or trimer phosphoramidites resulting in sequences containing nucleotide trimers.

An embodiment of the invention describes a synthetic DNA library or a member of a synthetic DNA library of antibodies or fragments of antibody molecules having heavy chains and lacking light chains wherein the CDR regions within variable domains of the heavy chains are synthesized using randomly assembled trimer phosphoramidites to eliminate unwanted cysteine amino acids and/or stop codons.

An embodiment of the invention is a method of preparing a synthetic single domain VHH library, comprising preparing synthetic sequences corresponding to DNA of four framework regions (FR1-FR4) of a single domain VHH antibody, preparing synthetic sequences of three complementarity determining region sequences (CDR1-CDR3) of said VHH antibody by random assembly of nucleotide trimers wherein said synthetic sequences contain sequences complementary to the ends of adjacent framework regions for hybridization to said adjacent framework regions, and assembling a synthetic DNA library encoding VHH antibodies using said synthetic DNA framework regions and said synthetic random nucleotide trimer sequences of said CDR regions containing said complementary ends to said framework regions.

An embodiment of the invention is a synthetic DNA library, comprising nucleotide sequences coding for a fragment of an immunoglobulin, said immunoglobulin fragment encoding for one or more single domain variable regions of heavy chain antibody devoid of light chain, wherein the sequences coding for the complementarity determining regions of said heavy chain variable region are synthesized randomly using trimer phosphoramidites.

An embodiment of the invention is a member of a synthetic DNA library, comprising nucleotide sequences coding for a fragment of an immunoglobulin, said immunoglobulin fragment comprising one or more single domain variable regions of heavy chain antibody devoid of light chain, wherein the sequences coding for the complementarity determining regions of said heavy chain variable region are synthesized randomly using trimer phosphoramidites.

### BRIEF DESCRIPTION OF THE DRAWINGS

FIGS. 1A-1B show a graphical description of the complete Sequence of a Camelid VHH Library for mRNA Display Showing Domains.

FIG. 2 illustrates, in schematic form, the oligonucleotides that were synthesized to assemble the synthetic VHH library. SynL1B (SEQ ID No. 1) contains a T7 promoter sequence and translation enhancer sequences that extend into SynL13 (SEQ ID No. 8).

FIG. 3 illustrates, in schematic form, the double-stranded segments of the library that were assembled from the single-stranded DNA oligonucleotides.

FIG. 4 illustrates the step-wise extension of the VHH library by addition of each double-stranded segment to the VHH construct.

FIG. 5 shows agarose gels in which partially assembled VHH library DNA was resolved.

FIG. 6 shows agarose gels of full length VHH DNA from an assembly reaction and after additional PCR amplification.

### DETAILED DESCRIPTION

The invention summarized above may be better understood by referring to the following description, drawings, and claims. This description of an embodiment, set out below to enable one to practice an implementation of the invention, is not intended to limit the preferred embodiment, but to serve as a particular example thereof. Those skilled in the art should appreciate that they may readily use the conception and specific embodiments disclosed as a basis for modifying or designing other methods and systems for carrying out the same purposes of the present invention. Those skilled in the art should also realize that such equivalent assemblies do not depart from the spirit and scope of the invention in its broadest form.

Single domain antibodies, single domain VHH, VHH Fragments or VHH antibodies, as used herein interchangeably, are antibody fragments comprised of the variable region or variable domain of single domain heavy chain only antibodies. Variable regions and variable domain are also herein used interchangeably. Traditional antibodies are composed of four polypeptides: two heavy chains and two light chains with a total molecular weight of about 150 kD.

A few species, notably camelids and some cartilaginous fish, produce an alternate heavy chain antibody that has two heavy chains and no light chains (Davies and Riechmann 1996). Several natural mutations or modifications in the antibody make this possible. One modification is that the heavy chain antibody mRNA is spliced to remove the third constant region of the heavy chain that is responsible for binding to light chain (Muyldermans et al. 2009). Hydrophobic to hydrophilic mutations of amino acids that are exposed due to the lack of a light chain, improve solubility of the heavy chain antibodies and reduce the tendency to aggregate (Harmsen and De Haard 2007; Muyldermans 2001). Antibody fragments comprised of the variable region of single domain heavy chain only antibodies are called VHH fragments or VHH antibodies. VHH antibodies have a molecular weight approximately 1/10^{th} that of full four-chain antibodies. In spite of the lack of light chain, VHH single domain antibodies can have high antigen binding affinity (Revets, De Baetselier, and Muyldermans 2005). This may be partly due to a longer CDR3 (complementarity determining or variable region 3) (Harmsen et al. 2000). “‘Complementarity determining regions’ (CDRs) are regions within antibodies (also known as immunoglobulins) or T cell receptors where these proteins complement an antigen's shape. Thus, CDRs determine the protein's affinity and specificity for specific antigens. The CDRs are the most variable part of the molecule, and contribute to the diversity of these molecules, allowing the antibody or the T cell receptor to recognize a vast repertoire of antigens” (Wikipedia definition). CDRs are sometimes called hypervariable regions.

In addition to the small size and potential for high affinity, camelid single domain VHH fragments have other advantages. They are soluble, stable to heat and pH, easy to select, and because VHH fragments and fusion proteins of VHH fragments are expressed as one polypeptide they are easy to manipulate and produce (Harmsen and De Haard 2007). In addition, they are poorly immunogenic because of high homology to human Vh and can be made even more homologous and less immunogenic with a few amino acid changes in the framework regions (Vincke et al. 2009).

Described is an invention that provides a method for development of single domain VHH antibodies (VHH fragments) using completely synthetic DNA sequences in the CDR regions of variable domains of the single domain VHH antibodies. As used herein, “synthetic DNA sequence” refers to a DNA sequence that has been developed utilizing the method described in this application, namely, synthesizing variable VHH regions using randomly assembled trinucleotide phosphoramidites. A “synthetic DNA library” means a DNA library that includes synthetic DNA sequences as described above.

As described above, previous methods of obtaining libraries of single domain antibodies required starting with libraries of antibody sequences obtained from B cells of live animals. This invention eliminates that need by using completely synthetic sequences. It is inefficient to simply use random nucleotides in the synthesis of variable regions because the percent of stop codons in the sequence would be statistically high enough to make the yield of full protein impractically low. In addition, it is often desirable to eliminate selected amino acids such as cysteines that can cause unwanted internal crosslinking. This invention provides a method for eliminating unwanted stop codons in the random variable region sequences by synthesizing variable regions using randomly assembled trinucleotide phosphoramidites to eliminate stop codons. The method also can be used, as desired, to eliminate any natural amino acid. It may be especially advantageous to eliminate trimers encoding cysteine to reduce unwanted crosslinking Natural amino acids are any amino acid used by any species. This method also has the positive effect of reducing frame-shift errors because nucleotides dropped during synthesis would have the effect of simply eliminating one amino acid while avoiding the undesirable frame-shift error seen if a single nucleotide were unincorporated.

In one aspect of the invention, the polypeptide may be based on single domain camelid antibody variable regions (VHH) having a backbone of four synthetic framework regions (FRs) and three variable domains (called CDRs) with random sequences. Variable regions of antibodies of the present invention are made using randomly assembled trinucleotide phosphoramidites. The randomly assembled trinucleotide phosphoramidites are mixed without stop codons. The randomly assembled trinucleotide phosphoramidites can be mixed without codons encoding cysteine. These two eliminations remove unwanted cysteine amino acids and stop codons. The random sequences could be combined with the fixed sequence framework regions using combinatorial PCR or other known molecular biological methods. The length of each CDR varies among natural camelid antibodies and could be varied from batch to batch in length for the synthetic random CDR regions in VHH fragments of the present invention. In one embodiment of the invention, CDR1 regions vary from 2 to 20 amino acids in length (6 to 60 nucleotides); CDR2 regions vary from 2 to 20 amino acids (6 to 60 nucleotides) and CDR3 regions from 4 to 40 amino acids (12 to 120 nucleotides). CDR 3 is usually longer that CDR 1 or CDR 2. In a preferred embodiment of the invention, CDR1 regions vary from 5 to 10 amino acids in length (15 to 30 nucleotides); CDR2 regions vary from 5 to 15 amino acids (15 to 45 nucleotides) and CDR3 regions from 8 to 24 amino acids (24 to 72 nucleotides); CDR 3 is usually longer than CDR 1 or CDR 2. Certain applications, such as the need to bind to deep parts of a target molecule, may require longer CDR3 regions.

In another aspect of the invention the targeting molecule may have one or more variable regions, each flanked by fixed non-antibody sequences that provide a framework structure different than antibody.

It is desirable to be able to select a member of the synthetic DNA library that encodes a VHH antibody having a desirable affinity, antigen specificity or any other desired property. In one exemplary embodiment of the present invention, mRNA display may be used to isolate molecules that bind to immobilized proteins or to proteins on cells and to simultaneously recover the polynucleotide encoding the binding molecules. mRNA display is one of several display or selection techniques that bind a protein to the nucleotide sequence encoding that protein. Other known selections techniques that bind a protein to the nucleotide sequence encoding that protein may be used. Examples of other selection techniques are phage display, yeast two-hybrid system, ribosome display and others.

In one aspect of the invention, selection of desired proteins is done in iterative cycles using the following sequence of events: 1) synthesis of a DNA library comprised of backbone sequences with synthetic variable regions, 2) PCR synthesis and assembly of a library with protein encoding sequences and other sequences such as promoter sequences and linker sequences as needed to do the subsequent steps of the procedure, 3) mRNA in vitro transcription, 4) hybridization of the in vitro transcribed mRNA to a linker also hybridizing to an oligonucleotide terminated at the 3′ terminus with puromycin, 5) in vitro translation with puromycin incorporation to create protein/mRNA complexes, 6) reverse transcription of mRNA to make protein/cDNA complexes, 7) optionally binding to non-target cells or tissues to remove unwanted binding proteins, 8) binding to target cells, 9) recovery of bound protein/cDNA complexes, and 9) PCR amplification of recovered DNA.

In one aspect of the invention, a camelid single domain VHH library may be assembled in the following manner: A library of synthetic camelid VHH antibodies may be constructed from overlapping, complementary oligonucleotides that may or may not have free phosphates on their 5′ ends. The overlapping oligonucleotides for the full length VHH library may be annealed and extended with a high fidelity, heat-resistant DNA polymerase by polymerase chain reaction (PCR) to join all of the oligonucleotides into full-length VHH constructs. The assembled library may be amplified by PCR and gel purified to remove unincorporated oligonucleotides and potential partial PCR products. Synthetic sequences for the four framework regions (FR1-FR4) may be based on consensus sequences as determined by alignment of known camelid antibody sequences. The three complementarity determining region sequences (CDR1-CDR3) may be generated by random assembly of nucleotide trimers using trimer phosphoramidites to form sequences of 12, 16, and 17 nucleotide trimers for CDR1, CDR2, and CDR3, respectively. Library constructs may include a T7 promoter and tobacco mosaic virus (TMV) translation enhancer region for efficient in vitro transcription and translation. A defined epitope tag such as FLAG or other known affinity tag may be included in the protein coding region to facilitate immunoisolation and visualization of the synthetic proteins. The 3′ end of the library construct preferably contains sequences to facilitate a physical connection between the transcribed RNA and nascent polypeptide chains via a puromycin-containing linker. The protein coding regions may not be terminated by a stop codon so that ribosomes may pause at the linker junction before disengaging from the mRNA.

In another aspect of the invention, a camelid single domain VHH library may be assembled in the following manner: Segments of the VHH library that each contain a single variable domain may be assembled from overlapping, complementary oligonucleotides that may or may not have free phosphates on their 5′ end. Each individual segments may share identical, overlapping sequences with the adjacent segments. The individually assembled, overlapping, double-stranded DNA segments may be joined together by combinatorial PCR using primers that anneal to the 5′ end of the upstream segment and the 3′ end of the downstream segment. The segments may be joined together in order from the upstream segment (containing CDR1) to the downstream segment (containing CDR3). Alternatively, the segments may be joined together in order from the downstream segment (containing CDR3) to the upstream segment (containing CDR1). Alternatively, the upstream segments may be joined together and the downstream segments may be joined together prior to assembly of the full-length VHH library.

In another aspect of the invention, a camelid single domain VHH library may be made in the following manner: Individually assembled, overlapping segments of the VHH library may be designed so that unique restriction sites are located within each overlapping non-variable domain. The unique restriction sites may be digested with the corresponding restriction enzyme and the digested DNA fragments may be gel-purified to remove small fragments of DNA. Digested segments with compatible ends may be mixed together and the compatible ends ligated together with DNA ligase prior to amplification of the joined segments by PCR.

In another aspect of the invention: Synthetic sequences for the four framework regions (FR1-FR4) may be based on consensus sequences as determined by alignment of known camelid antibody sequences. The three complementarity determining region sequences (CDR1-CDR3) may be generated by random assembly of nucleotide trimers to form sequences of 5-14, 6-18, and 8 to 22 nucleotide trimers for CDR1, CDR2, and CDR3, respectively. Library constructs may include a T7 promoter and tobacco mosaic virus (TMV) translation enhancer region for efficient in vitro transcription and translation. A defined epitope tag such as FLAG may be included in the protein coding region to facilitate immunoisolation and visualization of the synthetic proteins. The 3′ end of the library construct preferably contains sequences to facilitate a physical connection between the transcribed RNA and nascent polypeptide chains via a puromycin-containing linker. The protein coding regions may not be terminated by a stop codon so that ribosomes may pause at the linker junction before disengaging from the mRNA.

In another aspect of the invention: The length of the three complementarity determining region sequences (CDR1-CDR3) may be generated by random assembly of nucleotide trimers using trimer phosphoramidites to form sequences of 4 to 40 nucleotide trimers for each of CDR1, CDR2, and CDR3.

In another aspect of the invention: Individual VHH antibody genes that may be isolated from the library may be modified to contain a stop codon at the 3′ end of the protein coding region.

Example Sequences.

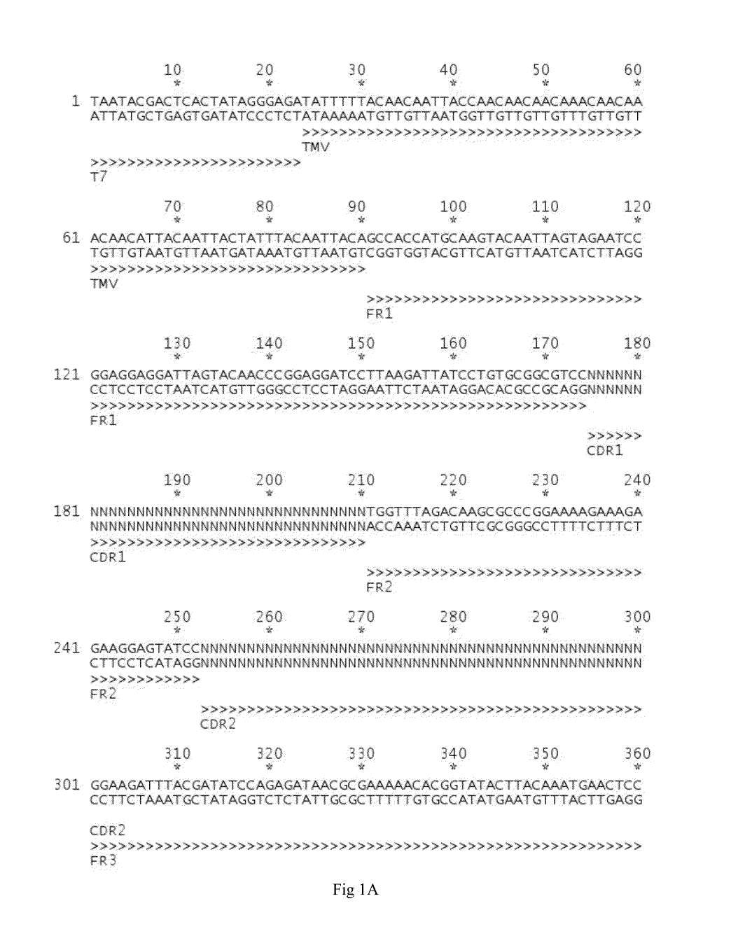

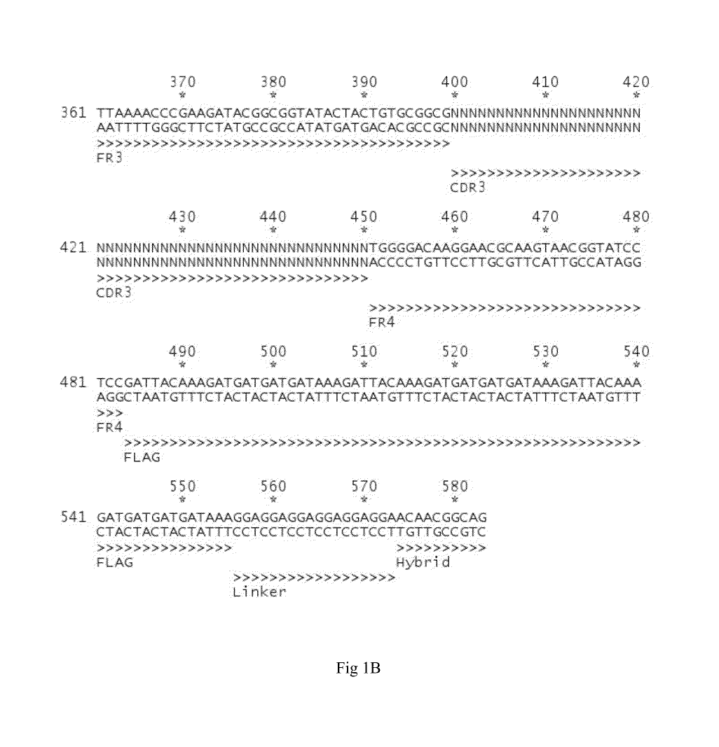

As shown in FIGS. 1A and 1B, DNA sequences for 65 natural camelid genes were obtained from Genbank. The sequences were aligned and consensus sequences for the framework regions (FR1-FR4) were determined according to the majority conserved residue at each position. A fully synthetic camelid antibody library sequence was designed based on the consensus sequence for each framework region. Random sequences were inserted for each of the complementarity determining regions (CDR1-CDR3). For the synthesis, each in-frame triplet NNN from the random sequence segments represents one of 19 trimer phosphoramidites that encode all of the natural amino acids except cysteine. The random sequences are composed of codon trimers assembled using trimer phosphoramidites to ensure that neither cysteine nor stop codons are incorporated into the full-length library. A T7 promoter sequence and TMV translation enhancer sequence were added to the 5′ end of the camelid VHH sequence. A FLAG tag (consisting of three repeats of the FLAG antigen sequence), a glycine spacer, and a sequence for annealing to a linker were added to the 3′ end of the synthetic camelid VHH sequence.

### Example 1

### One Method for Assembling a Synthetic Camelid VHH Library

Sequences for 65 natural camelid genes were obtained from GenBank. The sequences were aligned and consensus sequences for the framework regions were determined. A fully synthetic camelid antibody library sequence was designed using the consensus sequences for each framework region (FR1-FR4) and random sequences for the complementarity determining regions (CDR1-CDR3). A T7 promoter sequence and TMV translation enhancer sequence were added to the 5′ end of the camelid VHH sequence. A FLAG tag (consisting of three repeats of the FLAG antigen sequence), a glycine spacer, and a sequence for annealing to a linker were added to the 3′ end of the camelid VHH sequence. The DNA sequences for the repeated FLAG tags and glycine linkers were designed to utilize alternative codons for the amino acids in such a way that the DNA sequence does not repeat despite the repeats in the encoded amino acid sequence.

The full length VHH library was constructed from 19 DNA oligonucleotides that were synthesized by commercial vendors (Yale University Oligo Synthesis Resource and Integrated DNA Technologies). The trimer phosphoramidites for synthesis of the random regions were purchased from Glen Research. Eleven DNA oligonucleotides were synthesized that encode positive-strand segments of the VHH library sequence (SynL1B (SEQ ID No. 1), SynL13 (SEQ ID No. 8), SynL14 (SEQ ID No. 9), SynL24 (SEQ ID No. 15), SynL3 (SEQ ID No. 2), SynL26 (SEQ ID No. 17), SynL5 (SEQ ID No. 3), SynL28 (SEQ ID No. 19), SynL7 (SEQ ID No. 4), SynL30 (SEQ ID No. 20), and SynL31 (SEQ ID No. 21)). An additional 8 DNA oligonucleotides were synthesized that encode negative-strand sequence of the VHH library that were complementary and overlapping to the positive-strand oligonucleotides (SynL21 (SEQ ID No. 12), SynL22 (SEQ ID No. 13), SynL23 (SEQ ID No. 14), SynL25 (SEQ ID No. 16), SynL27 (SEQ ID No. 18), SynL32 (SEQ ID No. 22), SynL33 (SEQ ID No. 23), and SynL34 (SEQ ID No. 24)). The entire VHH library sequence is represented as either a positive or negative strand sequence. Gaps in the positive and negative strands are filled in by PCR during assembly of the library.

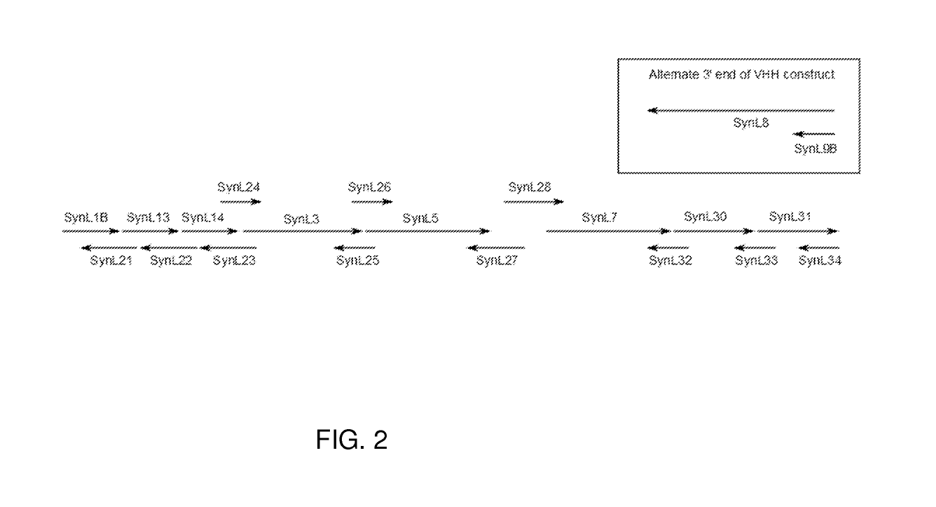

FIG. 2 illustrates, in schematic form, the oligonucleotides that were synthesized to assemble the synthetic VHH library. The positive-strand oligonucleotides SynL3 (SEQ ID No. 2), SynL5 (SEQ ID No. 3), and SynL7 (SEQ ID No. 4) contain the random sequences for CDR1, CDR2, and CDR3, respectively. The random sequences were synthesized with trimer phosphoramidites (purchased from Glen Research) to eliminate cysteines and stop codons. The remaining 16 oligonucleotides encode non-variable framework regions and flanking non-coding sequences. SynL1B (SEQ ID No. 1) contains a T7 promoter sequence and translation enhancer sequences that extend into SynL13 (SEQ ID No. 8). SynL14 (SEQ ID No. 9) contains the start codon for the VHH open reading frame. SynL30-SynL34 (SEQ ID No. 24) contain non-repeating DNA sequence that encode for the repeated amino acid sequences in the FLAG tag and glycine linker. SynL8 (SEQ ID No. 5) (see Example 2) contains an alternative, codon-optimized 3′ end which, as a result of the codon optimization, contains repeated DNA sequences. The mRNA display annealing sequences are identical in the two alternative 3′ ends.

Pilot lots of VHH library were constructed by assembling sub-segments of the library, then joining the smaller segments by combinatorial PCR. Four segments were assembled individually: 1) 5′ non-coding region plus FR1, 2) CDR2 flanked by FR1 and FR2 sequences, 3) CDR2 flanked by FR2 and FR3 sequences, and 4) CDR2 flanked by FR3 and FR4 sequences plus 3′ non-coding sequence. A fifth segment was assembled, but not used for the final construction, that contained only the FR4 and 3′ non-coding region of the VHH library.

Oligonucleotides were diluted in nuclease-free water to a working concentration of 10 μM. Assembly reactions for the pilot lots were carried out using high fidelity PCR (Q5 PCR kit, New England Biolabs) in 50 μA volumes with oligonucleotides at a final concentration of either 0.5 μM or 0.1 μm. The 5′ most and 3′ most oligonucleotides of each segment were added at the higher concentration; the internal oligonucleotides were added at the lower concentration. For example, Segment 1 was assembled in 50 μA reactions that contained 0.5 μM each of SynL1B (SEQ ID No. 1) and SynL23 (SEQ ID No. 14) plus 0.1 μM each of SynL13 (SEQ ID No. 8), SynL14 (SEQ ID No. 9), SynL21 (SEQ ID No. 12), and SynL22. Segment 2 was assembled in 50 μl reactions that contained 0.5 μM each of SynL24 (SEQ ID No. 15) and SynL25 (SEQ ID No. 16) plus 0.1 μM SynL3. Segment 3 was assembled in 50 μl reactions that contained 0.5 μM each of SynL26 (SEQ ID No. 17) and SynL27 (SEQ ID No. 18) plus 0.1 μM SynL5. Segment 4 was assembled in 50 μl reactions that contained 0.5 μM each of SynL28 (SEQ ID No. 19) and SynL34 (SEQ ID No. 24) plus 0.1 μM each of SynL7 (SEQ ID No. 4), SynL30 (SEQ ID No. 20), SynL31 (SEQ ID No. 21), SynL32 (SEQ ID No. 22), and SynL33 (SEQ ID No. 23).

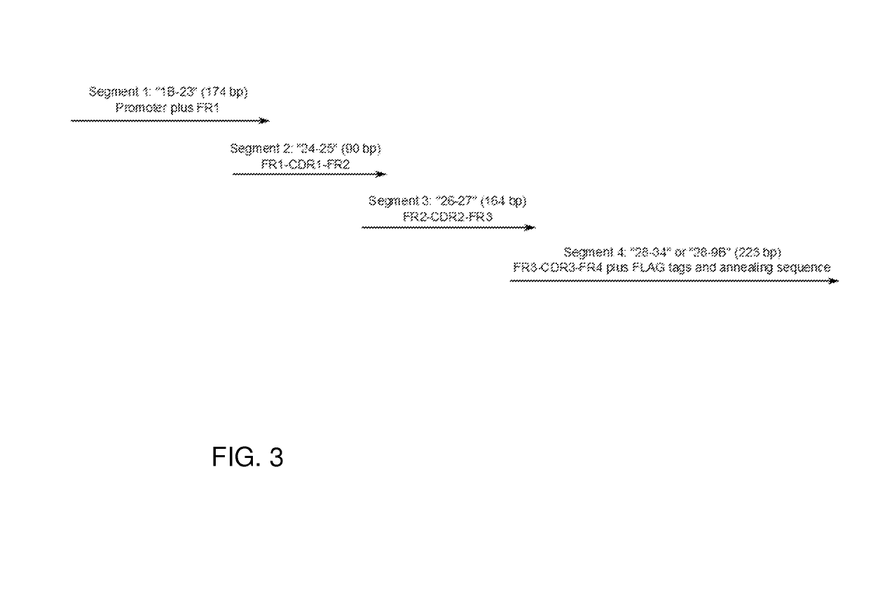

FIG. 3 illustrates, in schematic form, the double-stranded segments of the library that were assembled from the single-stranded DNA oligonucleotides. The individual DNA fragments are denoted by the oligonucleotides that are on the 5′ and 3′ end of each fragment: “1B-23 (SEQ ID No 25)”, “24-25 (SEQ ID No 28)”, “26-27 (SEQ ID No 29)”, and “28-34 (SEQ ID No 30)”.

Segment 1 (“1B-23 (SEQ ID No. 25)”) was joined with Segment 2 (“24-25 (SEQ ID No. 28)”) by high fidelity PCR in 50 μl reactions that contained 1 ng/μl each of Segment 1 DNA and Segment 2 DNA plus 0.5 μM each of SynL1B (SEQ ID No. 1) and SynL25 (SEQ ID No. 16) oligonucleotides. The resulting PCR product was gel purified and referred to as the “1B-25 (SEQ ID No. 26)” fragment. Segment 3 (“26-27 (SEQ ID No. 29)”) was attached to the “1B-25 (SEQ ID No. 26)” fragment by high fidelity PCR in 50 μl reactions that contained 1 ng/μl of each of Segment 3 DNA and “1B-25 (SEQ ID No. 26)” DNA plus 0.5 μM each of SynL1B (SEQ ID No. 1) and SynL27 (SEQ ID No. 18) oligonucleotides. The resulting PCR product was gel purified and referred to as the “1B-27 (SEQ ID No. 27)” fragment. The full-length VHH library, “1B-34” (SEQ ID No. 32), was assembled by joining Segment 4 (“28-34 (SEQ ID No. 30)”) to the “1B-27 (SEQ ID No. 27)” fragment by high fidelity PCR in 50 μA reactions that contained 1 ng/μl each of Segment 4 DNA and “1B-27 (SEQ ID No. 27)” DNA plus 0.5 μM each of SynL1B (SEQ ID No. 1) and SynL34 (SEQ ID No. 24) oligonucleotides. The full-length PCR product was gel purified and stored in nuclease-free water.

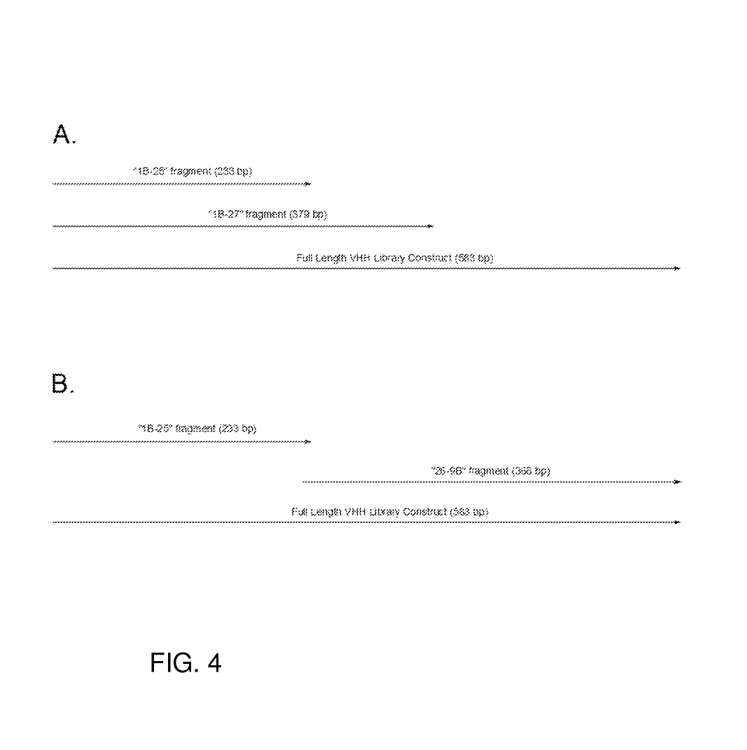

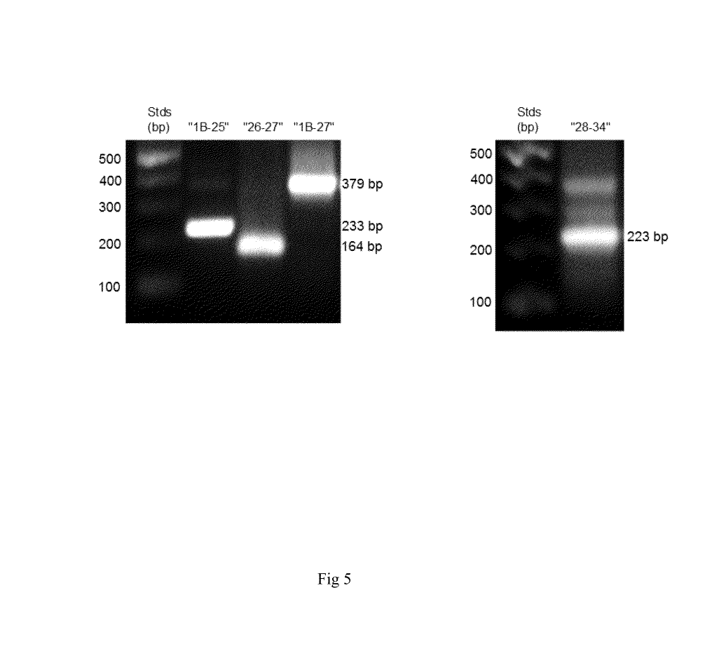

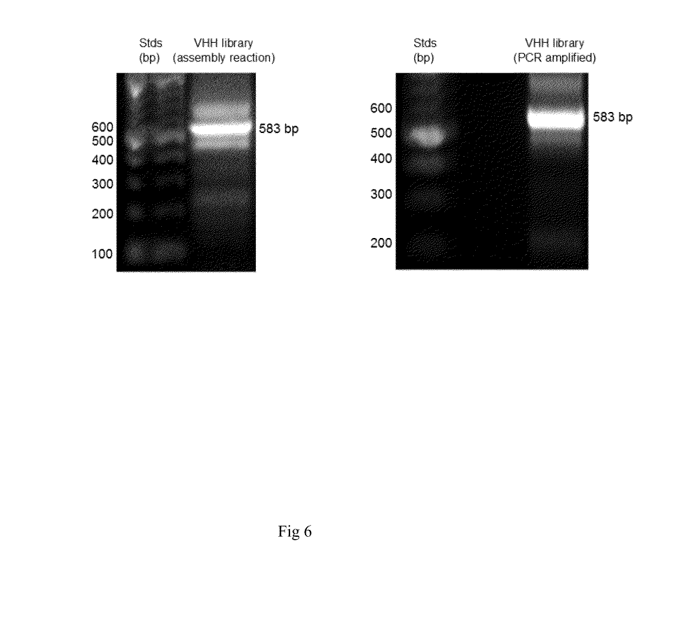

FIG. 4A illustrates the step-wise extension of the VHH library as described in Example 1. The “1B-25 (SEQ ID No. 26)” fragment is prepared by joining the “1B-23 (SEQ ID No. 25)” fragment with the “24-25 (SEQ ID No. 28)” fragment. The “1B-27 (SEQ ID No. 27)” fragment is made by joining the “26-27 (SEQ ID No. 29)” fragment to the “1B-25 (SEQ ID No. 26)” fragment. The full length construct is prepared by joining either the “28-34 (SEQ ID No. 30)” fragment or the “28-9B (SEQ ID No. 31)” fragment to the end of the “1B-27 (SEQ ID No. 27)” fragment. FIG. 5 shows agarose gels in which partially assembled VHH library DNA was resolved. FIG. 6 shows agarose gels of full length VHH DNA from an assembly reaction and after additional PCR amplification.

### Example 2

### An Alternative Method for Assembling a Synthetic Camelid VHH Library

Sequences for 65 natural camelid genes were obtained from GenBank. The sequences were aligned and consensus sequences for the framework regions were determined. A fully synthetic camelid antibody library sequence was designed using the consensus sequences for each framework region (FR1-FR4) and random sequences for the complementarity determining regions (CDR1-CDR3). A T7 promoter sequence and TMV translation enhancer sequence were added to the 5′ end of the camelid VHH sequence. A FLAG tag (consisting of three repeats of the FLAG antigen sequence), a glycine spacer, and a sequence for annealing to the PNA linker were added to the 3′ end of the camelid VHH sequence. The DNA sequences for the repeated FLAG tags and glycine linkers were codon-optimized to maximize protein translation efficiency. However, this resulted in repeated DNA sequences that reduced the efficiency of PCR amplification of full-length VHH library DNA. Conservative nucleotide changes were made in the codon-optimized sequences of FR2 and FR3 to introduce unique restriction enzyme sites (KasI and Bsu36I, respectively) in the DNA sequence without altering the consensus amino acid sequences in those regions. A unique restriction enzyme site, AflII, was fortuitously present at a useful location in FR1. These restriction enzyme sequences were chosen because they could not be generated by random assembly of the phosphoramidite trimers that were used to generate the random sequences.

The full length VHH library was constructed from 18 DNA oligonucleotides that were synthesized by commercial vendors (Yale University Oligo Synthesis Resource and Integrated DNA Technologies). Eleven DNA oligonucleotides were synthesized that encode positive-strand segments of the VHH library sequence (SynL1B (SEQ ID No. 1), SynL13 (SEQ ID No. 8), SynL14 (SEQ ID No. 9), SynL24 (SEQ ID No. 15), SynL3 (SEQ ID No. 2), SynL26 (SEQ ID No. 17), SynL5 (SEQ ID No. 3), SynL28 (SEQ ID No. 19), SynL7 (SEQ ID No. 4), SynL16 (SEQ ID No. 10), and SynL17 (SEQ ID No. 11)). An additional 7 DNA oligonucleotides were synthesized that encode negative-strand sequence of the VHH library that were complementary and overlapping to the positive-strand oligonucleotides (SynL21 (SEQ ID No. 12), SynL22 (SEQ ID No. 13), SynL23 (SEQ ID No. 14), SynL25 (SEQ ID No. 16), SynL27 (SEQ ID No. 18), SynL8 (SEQ ID No. 5), and SynL9B (SEQ ID No. 6)). The entire VHH library sequence was represented as either positive or negative strand sequence. Gaps in the positive and negative strands were filled in by PCR during assembly.

FIG. 2 illustrates, in schematic form, the oligonucleotides that were synthesized to assemble the synthetic VHH library. The positive-strand oligonucleotides SynL3 (SEQ ID No. 2), SynL5 (SEQ ID No. 3), and SynL7 (SEQ ID No. 4) contain the random sequences for CDR1, CDR2, and CDR3, respectively. The random sequences were synthesized with trimer phosphoramidites (purchased from Glen Research) to eliminate cysteines and stop codons. The remaining 15 oligonucleotides encode non-variable framework regions and flanking non-coding sequences. SynL1B (SEQ ID No. 1) contains a T7 promoter sequence and translation enhancer sequences that extend into SynL13 (SEQ ID No. 8). SynL14 (SEQ ID No. 9) contains the start codon for the VHH open reading frame. SynL25 (SEQ ID No. 16) and SynL26 (SEQ ID No. 17) contain the deviations from codon optimization that generate a unique KasI restriction site in FR2. SynL27 (SEQ ID No. 18) and SynL28 (SEQ ID No. 19) contain the deviations from codon optimization that generate a unique Bsu36I restriction site in FR3. SynL8 (SEQ ID No. 5) contains a codon-optimized 3′ end which, as a result of the codon optimization, contains repeated DNA sequences. SynL30-SynL34 (SEQ ID No. 20-24) (see Example 1) contain an alternative non-repeating DNA sequence that encode for the repeated amino acid sequences in the FLAG tag and glycine linker. The mRNA display annealing sequences are identical in the two alternative 3′ ends.

Pilot lots of VHH library were constructed by assembling sub-segments of the library, then joining the smaller segments by combinatorial PCR or restriction digestion and ligation followed by PCR amplification. Four segments were assembled individually: 1) 5′ non-coding region plus FR1, 2) CDR2 flanked by FR1 and FR2 sequences, 3) CDR2 flanked by FR2 and FR3 sequences, and 4) CDR2 flanked by FR3 and FR4 sequences plus 3′ non-coding sequence.

Oligonucleotides were diluted in nuclease-free water to a working concentration of 10 μM. Assembly reactions for the pilot lots were carried out using high fidelity PCR (Phusion PCR kit, New England Biolabs) in 50 μl volumes with oligonucleotides at a final concentration of either 0.5 μM or 0.1 μM. The 5′ most and 3′ most oligonucleotides of each segment were added at the higher concentration; the internal oligonucleotides were added at the lower concentration. For example, Segment 1 was assembled in 50 μl reactions that contained 0.5 μM each of SynL1B (SEQ ID No. 1) and SynL23 (SEQ ID No. 14) plus 0.1 μM each of SynL13 (SEQ ID No. 8), SynL14 (SEQ ID No. 9), SynL21 (SEQ ID No. 12), and SynL22. Segment 2 was assembled in 50 μl reactions that contained 0.5 μM each of SynL24 (SEQ ID No. 15) and SynL25 (SEQ ID No. 16) plus 0.1 μM SynL3. Segment 3 was assembled in 50 μl reactions that contained 0.5 μM each of SynL26 (SEQ ID No. 17) and SynL27 (SEQ ID No. 18) plus 0.1 μM SynL5. Segment 4 was assembled in 50 μl reactions that contained 0.5 μM each of SynL28 (SEQ ID No. 19) and SynL9B (SEQ ID No. 6) plus 0.1 μM each of SynL7 (SEQ ID No. 4) and SynL8 (SEQ ID No. 5).

FIG. 3 illustrates, in schematic form, the double-stranded segments of the library that were assembled from the single-stranded DNA oligonucleotides. The individual DNA fragments are denoted by the oligonucleotides that are on the 5′ and 3′ end of each fragment: “1B-23 (SEQ ID No. 25)”, “24-25 (SEQ ID No. 28)”, “26-27 (SEQ ID No. 29)”, and “28-34 (SEQ ID No. 30)”. Segment 1 (“1B-23 (SEQ ID No. 25)”) was joined with Segment 2 (“24-25 (SEQ ID No. 28)”) by high fidelity PCR in 50 μl reactions that contained 1 ng/μl each of Segment 1 DNA and Segment 2 DNA plus 0.5 μM each of SynL1B (SEQ ID No. 1) and SynL25 (SEQ ID No. 16) oligonucleotides. The resulting PCR product was gel purified and referred to as the “1-25” fragment. Segment 3 (“26-27 (SEQ ID No. 29)”) was joined to Segment 4 (“28-34 (SEQ ID No. 30)”) by restriction digestion, ligation, and PCR amplification. Approximately 0.5 μg of Segment 3 DNA and 0.5 μg of Segment 4 DNA were each digested with Bsu36I enzyme according to manufacturer's instructions (New England Biolabs). The digested DNA was gel purified and ½ of the recovered DNA (˜0.25 μg of each, 0.5 μg total DNA) of each were combined in a 30 μl ligation reaction using T4 DNA ligase that was incubated for 12 h at 16° C. Five microliters of ligation mixture (˜83 ng or 1.66 ng/μl DNA) was amplified by high fidelity PCR in a 50 μl reaction that contained 0.5 μM of each SynL26 (SEQ ID No. 17) and SynL9B (SEQ ID No. 6) oligonucleotides. The resulting PCR product was gel purified and referred to as the “26-9” fragment. The “1-25” fragment and “26-9” fragment were joined together by restriction digestion, ligation, and PCR amplification. Approximately 0.5 μg of “1-25” DNA and 0.5 μg of “26-9” DNA were digested with KasI enzyme according to manufacturer's instructions (New England Biolabs). The digested DNA was gel purified and ⅓ of the recovered material (˜0.133 μg of each, 266 ng total DNA) were combined in a 30 μl ligation reaction using T4 DNA ligase that was incubated for 12 h at 16° C. Five microliters of the ligation mixture (44.3 ng or 0.89 ng/μl DNA) was amplified by high fidelity PCR in a 50 μl reaction that contained 0.5 μM each of SynL1B (SEQ ID No. 1) and SynL9B (SEQ ID No. 6) oligonucleotides. The resulting full-length VHH PCR product was gel purified and stored in nuclease-free water.

FIG. 4B illustrates the step-wise assembly of the VHH library as described in Example 2. The “1B-25 (SEQ ID No. 26)” fragment is prepared by joining the “1B-23 (SEQ ID No. 25)” fragment with the “24-25 (SEQ ID No. 28)” fragment. The “26-9B” (SEQ ID No. 36) fragment is made by joining the “26-27 (SEQ ID No. 29)” fragment to the “28-9B (SEQ ID No. 31)” fragment. The full length construct, “1B-9B” (SEQ ID No. 33), is prepared by joining the “1B-25 (SEQ ID No. 26)” fragment with the “26-9B” (SEQ ID No. 36) fragment.

Full-length VHH library DNA was subcloned into a plasmid vector and representative samples were isolated and sequenced. The open reading frame for one isolated clone is presented as SEQ ID No. 34. The protein encoded by the isolated cloned VHH DNA is presented as SEQ ID No. 35. These data demonstrate that intact VHH genes may be assembled from single-stranded oligonucleotides following the methods described above.

For the display or selection techniques, substrates for affinity binding and antibody selection may be from several sources. In one example, human prostate cancer cell lines and tissue sections from human prostate cancer and other human tissues can be used. Human cell lines other than prostate cells and non-prostate tissues may be used for negative selections to remove antibodies that bind to non-target tissue. After isolating several of these antibodies, cDNAs encoding the antibodies may be cloned and sequenced to determine the diversity of the antibodies selected by this technique. Individual candidate antibodies may be purified and used in immunohistochemistry studies to identify where the antibodies bind within the target tissue. Antibodies that bind to targeted cells in tissue (in this example prostate cancer and normal prostate cells) may be retained while those that bind to non-target cells (for example epithelial cells, endothelial cells and fibroblasts) may be discarded or kept in a separate library. Any cancer cell line, non-cancer cell line or thin or thick sections of tissues may be substituted for the prostate cancer cells in this example. Cells or tissues from any species to include human may be used.

VHH fragments may be molecularly characterized in a number of ways and their cell and tissue binding patterns may be determined.

Molecular characterization of VHH fragments: Recovered DNA may be sub-cloned into plasmid vectors expressing an alkaline phosphatase-ligand fusion protein, and unique clones may be identified by restriction digestion. A representative of each unique clone may be sequenced and the sequences may be compared to identify any common motifs in the CDR regions. Ligand proteins may be produced by expression in E. coli and FLAG affinity purified. Specific binding to target cell lines may be confirmed for each individual clone. Receptors or other cell surface molecules that the VHH fragments bind to on cells may be identified by affinity purification of receptors using FLAG purified ligand proteins.

Identify binding distribution in tissues—specificity of binding: Specificity of binding of proteins identified using these procedures may be evaluated in a number of ways. One example is that antibody-alkaline phosphatase fusion proteins may be engineered to evaluate binding. This fusion protein may be used to evaluate binding to a panel of human or animal tissues. The tissue can be in the form of slides made from sections of fixed or frozen tissue. Binding can be identified using standard immunohistochemical techniques. Using prostate cancer as an example, tissues, arrays of tissues and frozen sections may be purchased from a company such as US Biomax (prostate cancer array paraffin, FDA 2 slide set, Normal tissue array (20 cases×2), Multiple cancers array, prostate cancer 24 cases (frozen sections)) or from the Prostate Cancer Biorepository (prostate cancer Frozen section slides, prostate cancer arrays (frozen section)). Of particular interest in this list is the array of frozen sections that meet FDA requirements for testing non-specific binding and the prostate cancer arrays (obtainable from both of the sources). Tissues may be incubated in the labeled ligand, washed and stained using insoluble substrate. Alkaline phosphatase labeled anti-actin antibody may be used as a method positive control. Antibodies selected above that bind to tissues other than prostate may also be used as controls.

Estimate Binding Affinity: Affinity of binding to cell surface targets may be determined using standard methods. Instruments such as the KinexA or the Biocore 2000 may be used with manufacturer's instructions. Alternatively a modified cell based ELISA (optical absorption, fluorescent or chemiluminescent) method may be used. For that method, the proteins may be expressed in E. coli and purified using FLAG affinity chromatography. Quantity and purity of the purified protein may be determined using standard methods. Before use in the assay, specific activity of the purified proteins and the linear range of signal may be determined. Dilutions of antibody (˜2-16 nM) may be incubated with a range of cell concentrations and incubated for 2 h at 4° C. After separating the cells by centrifugation, quantity of ligand on the cells and in the supernatant may be assayed by ELISA. Ligand affinity and an estimated number of cell surface receptors may be calculated using published mathematical methods. Alternatively, specialized commercial equipment such as those made by Biacore, Kinex or Forte Bio may be used to determine VHH fragment binding affinity using label free techniques.

Identify ligand receptors: Molecular weight and other properties of the cellular target (receptor) proteins may be determined using polyacrylamide gel electrophoresis and Western blot. Identity of cellular target proteins also may be determined. For this, affinity columns may be made using purified VHH fragments. Solubilized total cell proteins may be purified on the ligand affinity columns. Alternatively proteins previously identified using Western blot may be purified using 2 D gel electrophoresis. A combination of size exclusion, electrophoresis, and affinity purification may be used to acquire purified proteins. However obtained, the purified proteins may be sent to contract labs for Edman sequencing and mass spectroscopy to determine their identities.

Examples for making camelid antibodies using synthetic CDR regions or other VHH fragments or ligands are not limiting. None of these examples are meant to be limiting.

The invention has been described with references to a preferred embodiment. While specific values, relationships, materials and steps have been set forth for purposes of describing concepts of the invention, it will be appreciated by persons skilled in the art that numerous variations and/or modifications may be made to the invention as shown in the specific embodiments without departing from the spirit or scope of the basic concepts and operating principles of the invention as broadly described. It should be recognized that, in the light of the above teachings, those skilled in the art can modify those specifics without departing from the invention taught herein. Having now fully set forth the preferred embodiments and certain modifications of the concept underlying the present invention, various other embodiments as well as certain variations and modifications of the embodiments herein shown and described will obviously occur to those skilled in the art upon becoming familiar with such underlying concept. It is intended to include all such modifications, alternatives and other embodiments insofar as they come within the scope of the appended claims or equivalents thereof. It should be understood, therefore, that the invention may be practiced otherwise than as specifically set forth herein. Consequently, the present embodiments are to be considered in all respects as illustrative and not restrictive.

### REFERENCES

The references cited in this application are incorporated herein by reference in their entirety.
Davies, J, and L Riechmann. 1996. “Single Antibody Domains as Small Recognition Units: Design and in Vitro Antigen Selection of Camelized, Human VH Domains with Improved Protein Stability.” Protein Engineering 9 (6) (June): 531-537.
Harmsen, M M, and H J De Haard. 2007. “Properties, Production, and Applications of Camelid Single-domain Antibody Fragments.” Applied Microbiology and Biotechnology 77 (1) (November): 13-22. doi:10.1007/s00253-007-1142-2.
Harmsen, M M, R C Ruuls, I J Nijman, T A Niewold, L G Frenken, and B de Geus. 2000. “Llama Heavy-chain V Regions Consist of at Least Four Distinct Subfamilies Revealing Novel Sequence Features.” Molecular Immunology 37 (10) (August): 579-590.
Muyldermans, S. 2001. “Single Domain Camel Antibodies: Current Status.” Journal of Biotechnology 74 (4) (June): 277-302.
Muyldermans, S, T N Baral, V Cortez Retamozzo, P De Baetselier, E De Genst, J Kinne, H Leonhardt, et al. 2009. “Camelid Immunoglobulins and Nanobody Technology.” Veterinary Immunology and Immunopathology 128 (1-3) (March 15): 178-183. doi:10.1016/j.vetimm.2008.10.299.
Muyldermans, S, and M Lauwereys. 1999. “Unique Single-domain Antigen Binding Fragments Derived from Naturally Occurring Camel Heavy-chain Antibodies.” Journal of Molecular Recognition: JMR 12 (2) (April): 131-140. doi:10.1002/(SICI)1099-1352(199903/04)12:2<131::AID-JMR454>3.0.CO;2-M.
Revets, Hilde, Patrick De Baetselier, and Serge Muyldermans. 2005. “Nanobodies as Novel Agents for Cancer Therapy.” Expert Opinion on Biological Therapy 5 (1) (January): 111-124. doi:10.1517/14712598.5.1.111.
Van der Linden, R H, B de Geus, G J Frenken, H Peters, and C T Verrips. 2000. “Improved Production and Function of Llama Heavy Chain Antibody Fragments by Molecular Evolution.” Journal of Biotechnology 80 (3) (July 14): 261-270.
Vincke, Cëcile, Remy Loris, Dirk Saerens, Sergio Martinez-Rodriguez, Serge Muyldermans, and Katja Conrath. 2009. “General Strategy to Humanize a Camelid Single-domain Antibody and Identification of a Universal Humanized Nanobody Scaffold.” The Journal of Biological Chemistry 284 (5) (January 30): 3273-3284. doi:10.1074/jbc.M806889200.

What is claimed is:

1. A method of preparing a synthetic single domain VHH library, comprising:
A. preparing synthetic sequences corresponding to cDNA of four framework regions (FR1-FR4) of a single domain VHH antibody,
B. preparing synthetic sequences of three complementarity determining region sequences (CDR1-CDR3) of said VHH antibody by random assembly of nucleotide trimers wherein said synthetic sequences contain sequences complementary to the ends of adjacent framework regions for hybridization to said adjacent framework regions, and
C. assembling a synthetic DNA library encoding VHH antibodies using said synthetic cDNA framework regions and said synthetic random nucleotide trimer sequences of said CDR regions with said complementary ends to said framework regions.

2. The method of claim 1, further comprising one or more sequences selected from the group consisting of a eukaryotic promoter, a prokaryotic promoter, a promoter enhancer.

3. The method of claim 1, further comprising one or more sequences of a linker, and an affinity recognition site.

4. The method of claim 1 wherein said synthetic sequences corresponding to cDNA each of the four framework regions are consensus sequences of several framework regions of each of the corresponding FR1-FR4.

5. A synthetic DNA library, comprising:
nucleotide sequences coding for a fragment of an immunoglobulin, said immunoglobulin fragment encoding for one or more single domain variable regions of heavy chain antibody devoid of light chain, wherein
the sequences coding for the complementarity determining regions of said heavy chain variable region are synthesized randomly using trimer phosphoramidites.

6. The synthetic DNA library of claim 5 wherein said random synthesis is done using a mixture of trimer phosphoramidites devoid of sequences encoding stop codons.

7. The synthetic DNA library of claim 5 wherein said random synthesis is done using a mixture of trimer phosphoramidites devoid of sequences encoding cysteine or any other selected natural amino acid.

8. A member of a synthetic DNA library, comprising:
nucleotide sequences coding for a fragment of an immunoglobulin, said immunoglobulin fragment comprising one or more single domain variable regions of heavy chain antibody devoid of light chain, wherein
the sequences coding for the complementarity determining regions of said heavy chain variable region are synthesized randomly using trimer phosphoramidites.

9. The member of a DNA library of claim 8 wherein said random synthesis is done using a mixture of trimer phosphoramidites devoid of sequences encoding stop codons.

10. The member of a DNA library of claim 8 wherein said random synthesis is done using a mixture of trimer phosphoramidites devoid of sequences encoding cysteine or any other selected natural amino acid


SEQUENCE LISTING (54 sequences)

SEQ ID NO: 1 - DNA, 45 bp, Artificial Sequence
  Synthetic Oligonucleotide
taatacgactcactatagggagatatttttacaacaattaccaac

SEQ ID NO: 2 - DNA, 25 bp, Artificial Sequence
  Synthetic Oligonucleotide
cttaagattatcctgtgcggcgtcc

SEQ ID NO: 3 - DNA, 23 bp, Artificial Sequence
  Synthetic Oligonucleotide
gaaaagaaagagaaggagtatcc

SEQ ID NO: 4 - DNA, 20 bp, Artificial Sequence
  Synthetic Oligonucleotide
cggtatactactgtgcggcg

SEQ ID NO: 5 - DNA, 133 bp, Artificial Sequence
  Synthetic Oligonucleotide
ctgccgttgttcctcctcctcctcctcctttatcatcatcatctttgtaatctttatcatcatcatctttgtaatctttatcatcatcatctttgtaatcggaggataccgttacttgcgttccttgtcccca

SEQ ID NO: 6 - DNA, 34 bp, Artificial Sequence
  Synthetic Oligonucleotide
ctgccgttgttcctcctcctcctcctcctttatc

SEQ ID NO: 7 - DNA, 24 bp, Artificial Sequence
  Synthetic Oligonucleotide
tcctcctcctcctcctcctttatc

SEQ ID NO: 8 - DNA, 51 bp, Artificial Sequence
  Synthetic Consensus Sequence from Lama pacos and Lama glama
aacaacaaacaacaaacaacattacaattactatttacaattacagccacc

SEQ ID NO: 9 - DNA, 53 bp, Artificial Sequence
  Synthetic Consensus Sequence from Lama pacos and Lama glama
atgcaagtacaattagtagaatccggaggaggattagtacaacccggaggatc

SEQ ID NO: 10 - DNA, 55 bp, Artificial Sequence
  Synthetic Consensus Sequence from Lama pacos and Lama glama
ggtatcctccgattacaaagatgatgatgataaagattacaaagatgatgatgat

SEQ ID NO: 11 - DNA, 55 bp, Artificial Sequence
  synthetic oligonucleotide
aaagattacaaagatgatgatgataaaggaggaggaggaggaggaacaacggcag

SEQ ID NO: 12 - DNA, 56 bp, Artificial Sequence
  Synthetic Consensus Sequence from Lama pacos and Lama glama
tgtaatgttgtttgttgtttgttgttgttggtaattgttgtaaaaatatctcccta

SEQ ID NO: 13 - DNA, 52 bp, Artificial Sequence
  Synthetic Consensus Sequence from Lama pacos and Lama glama
tccggattctactaattgtacttgcatggtggctgtaattgtaaatagtaat

SEQ ID NO: 14 - DNA, 51 bp, Artificial Sequence
  Synthetic Consensus Sequence from Lama pacos and Lama glama
ggacgccgcacaggataatcttaaggatcctccgggttgtactaatcctcc

SEQ ID NO: 15 - DNA, 31 bp, Artificial Sequence
  Synthetic Consensus Sequence from Lama pacos and Lama glama
aggatccttaagattatcctgtgcggcgtcc

SEQ ID NO: 16 - DNA, 23 bp, Artificial Sequence
  Synthetic Consensus Sequence from Lama pacos and Lama glama
tttccgggcgcctgtctaaacca

SEQ ID NO: 17 - DNA, 37 bp, Artificial Sequence
  Synthetic Consensus Sequence from Lama pacos and Lama glama
tagacaggcgcccggaaaagaaagagaaggagtatcc

SEQ ID NO: 18 - DNA, 79 bp, Artificial Sequence
  Synthetic Consensus Sequence from Lama pacos and Lama glama
ccgtatcctcaggttttaaggagttcatttgtaagtataccgtgtttttcgcgttatctctggatatcgtaaatcttcc

SEQ ID NO: 19 - DNA, 39 bp, Artificial Sequence
  Synthetic Consensus Sequence from Lama pacos and Lama glama
ttaaaacctgaggatacggcggtatactactgtgcggcg

SEQ ID NO: 20 - DNA, 55 bp, Artificial Sequence
  Synthetic Consensus Sequence from Lama pacos and Lama glama
ggtatcctccgattacaaagatgatgatgataaagactacaaggatgacgatgac

SEQ ID NO: 21 - DNA, 55 bp, Artificial Sequence
  shynthetic construct for VHH library with random trimers
aaagactataaggacgacgacgacaagggaggtggcggtggagggacaacggcag

SEQ ID NO: 22 - DNA, 32 bp, Artificial Sequence
  Synthetic Consensus Sequence from Lama pacos and Lama glama
gtaatcggaggataccgttacttgcgttcctt

SEQ ID NO: 23 - DNA, 32 bp, Artificial Sequence
  synthetic oligonucleotide
cgtccttatagtctttgtcatcgtcatccttg

SEQ ID NO: 24 - DNA, 33 bp, Artificial Sequence
  synthetic oligonucleotide
ctgccgttgtccctccaccgccacctcccttgt

SEQ ID NO: 25 - DNA, 174 bp, Artificial Sequence
  VHH Fragment
taatacgactcactatagggagatatttttacaacaattaccaacaacaacaaacaacaaacaacattacaattactatttacaattacagccaccatgcaagtacaattagtagaatccggaggaggattagtacaacccggaggatccttaagattatcctgtgcggcgtcc

SEQ ID NO: 26 - DNA, 174 bp, Artificial Sequence; identical to SEQ ID NO: 25
  VHH Fragment

SEQ ID NO: 27 - DNA, 174 bp, Artificial Sequence; identical to SEQ ID NO: 25
  VHH Fragment

SEQ ID NO: 28 - DNA, 31 bp, Artificial Sequence; identical to SEQ ID NO: 15
  VHH Fragment

SEQ ID NO: 29 - DNA, 37 bp, Artificial Sequence
  VHH Fragment
tagacaagcgcccggaaaagaaagagaaggagtatcc

SEQ ID NO: 30 - DNA, 39 bp, Artificial Sequence
  VHH Fragment
ttaaaacccgaagatacggcggtatactactgtgcggcg

SEQ ID NO: 31 - DNA, 39 bp, Artificial Sequence; identical to SEQ ID NO: 30
  VHH Fragment

SEQ ID NO: 32 - DNA, 174 bp, Artificial Sequence; identical to SEQ ID NO: 25
  VHH Fragment with random trimers

SEQ ID NO: 33 - DNA, 174 bp, Artificial Sequence; identical to SEQ ID NO: 25
  VHH Fragment with random trimers

SEQ ID NO: 34 - DNA, 211 bp, Artificial Sequence
  Synthetic with Lama concensus sequences
atgcaagtacaattagtagaatccggaggaggattagtacaacccggaggatccttaagattatcctgtgcggcgtcctggggacaaggaacgcaagtaacggtatcctccgattacaaagatgatgatgataaagattacaaagatgatgatgataaagattacaaagatgatgatgataaaggaggaggaggaggaggaacaacggcag

SEQ ID NO: 35 - protein, 70 aa, Artificial Sequence
  Synthetic consensus with Lama sequence
MQVQLVESGGGLVQPGGSLRLSCAASWGQGTQVTVSSDYKDDDDKDYKDDDDKDYKDDDDKGGGGGGTTA

SEQ ID NO: 36 - DNA, 37 bp, Artificial Sequence; identical to SEQ ID NO: 29
  VHH Fragment

SEQ ID NO: 37 - DNA, 19 bp, Artificial
  Synthetic
tggtttagacaagcgcccg

SEQ ID NO: 38 - DNA, 20 bp, Artificial
  Synthetic
ggaagatttacgatatccag

SEQ ID NO: 39 - DNA, 23 bp, Artificial
  Synthetic
tggggacaaggaacgcaagtaac

SEQ ID NO: 40 - DNA, 23 bp, Artificial
  Synthetic
tggtttagacaagcgcccggaaa

SEQ ID NO: 41 - DNA, 42 bp, Artificial
  Synthetic
tggtttagacaagcgcccggaaaagaaagagaaggagtatcc

SEQ ID NO: 42 - DNA, 79 bp, Artificial
  Synthetic
ggaagatttacgatatccagagataacgcgaaaaacacggtatacttacaaatgaactccttaaaacccgaagatacgg

SEQ ID NO: 43 - DNA, 23 bp, Artificial
  Synthetic
tggtttagacaagcgcccggaaa

SEQ ID NO: 44 - DNA, 79 bp, Artificial; identical to SEQ ID NO: 42
  synthetic

SEQ ID NO: 45 - DNA, 133 bp, Artificial
  Synthetic
tggggacaaggaacgcaagtaacggtatcctccgattacaaagatgatgatgataaagactacaaggatgacgatgacaaagactataaggacgacgacgacaagggaggtggcggtggagggacaacggcag

SEQ ID NO: 46 - DNA, 133 bp, Artificial
  Synthetic
tggggacaaggaacgcaagtaacggtatcctccgattacaaagatgatgatgataaagattacaaagatgatgatgataaagattacaaagatgatgatgataaaggaggaggaggaggaggaacaacggcag

SEQ ID NO: 47 - DNA, 42 bp, Artificial; identical to SEQ ID NO: 41
  Synthetic

SEQ ID NO: 48 - DNA, 99 bp, Artificial
  Synthetic
ggaagatttacgatatccagagataacgcgaaaaacacggtatacttacaaatgaactccttaaaacccgaagatacggcggtatactactgtgcggcg

SEQ ID NO: 49 - DNA, 133 bp, Artificial; identical to SEQ ID NO: 45
  Synthetic

SEQ ID NO: 50 - DNA, 42 bp, Artificial; identical to SEQ ID NO: 41
  Synthetic

SEQ ID NO: 51 - DNA, 99 bp, Artificial; identical to SEQ ID NO: 48
  Synthetic

SEQ ID NO: 52 - DNA, 133 bp, Artificial; identical to SEQ ID NO: 46
  Synthetic

SEQ ID NO: 53 - DNA, 99 bp, Artificial; identical to SEQ ID NO: 48
  Synthetic

SEQ ID NO: 54 - DNA, 133 bp, Artificial; identical to SEQ ID NO: 45
  Synthetic